(19) United States
(12) Patent Application Publication
(10) Pub. No.: US 2012/0271430 A1
(43) Pub. Date: Oct. 25, 2012

(54) ANKLE ARTHROPLASTY

(75) Inventors: Jeffery D. Arnett, Gilbert, AZ (US); Joshua A. Butters, Chandler, AZ (US); Dylan M. Hushka, Gilbert, AZ (US)

(73) Assignees: MEDICINELODGE, INC. DBA IMDS CO-INNOVATION, Logan, UT (US); Neil Duggal, London (CA)

(21) Appl. No.: 13/452,692

(22) Filed: Apr. 20, 2012

Related U.S. Application Data
(60) Provisional application No. 61/478,254, filed on Apr. 22, 2011.

(51) Int. Cl. A61F 2/42

(52) U.S. Cl. 623/21.18

(57) ABSTRACT

Total ankle arthroplasty with a tibial plate, a talar plate and a middle or core component. The ankle arthroplasty may allow for varus or valgus accommodation through the use of a core component with various medial and lateral heights in varus and valgus orientations. In addition the resurfacing of the talus is accomplished with a talar plate with a curved orientation that is congruent to one surface of the core component to allow for appropriate ankle manipulation.

### CROSS-REFERENCE TO RELATED APPLICATIONS

This application claims the benefit of the following which is incorporated herein by reference:

U.S. Provisional Patent Application No. 61/478,254, filed Apr. 22, 2011, entitled TOTAL ANKLE ARTHROPLASTY WITH VARUS-VALGUS ACCOMMODATION, Attorney's docket no. DUG-11 PROV, which is pending.

### BACKGROUND

The ankle, or talocrural joint, is a synovial hinge joint that connects the distal ends of the tibia and fibula in the lower limb with the proximal end of the talus bone in the foot. This joint plays an integral role in balance, muscle stabilization, load bearing and motion, and is responsible for the upwards and downwards movement of the foot. Total ankle replacement is often necessary for patients with arthritis or other degenerative or traumatic conditions. Often when choosing a total ankle replacement system, a varus-valgus design is desirable to accommodate different patient deformities.

The present disclosure relates to systems, apparatus, method and kit for total joint replacement. Specifically, this disclosure relates to a total ankle replacement apparatus, system, kit and methods suitable to accommodate or correct various patient deformities. The disclosed ankle replacement may resist off center loads by restricting some of the degrees of freedom of rotation. This resistance may result from an alignment system in which a component contains a slot in which a rib of an endplate slides. The ability to resist off center loads may allow the disclosed ankle replacement to accommodate issues such as various patient deformities and different surgical placement procedures. By adjusting the varus-valgus orientation of a core piece of the ankle replacement system, the disclosed system may provide stability to the weight bearing ankle joint in patients with various deformities.

While the examples in the present disclosure relate to the ankle joint, the systems and methods are applicable to other synovial joints in the body.

### BRIEF DESCRIPTION OF THE DRAWINGS

Various examples of the present technology will now be discussed with reference to the appended drawings. It is appreciated that these drawings depict only typical examples of the technology and are therefore not to be considered limiting of its scope.

FIG. 1 is a perspective assembly view of an ankle replacement system;

FIG. 2A is a top perspective view of a tibial endplate of the system of FIG. 1;

FIG. 2B is a bottom perspective view of the tibial endplate of FIG. 2A;

FIG. 3A is a perspective top view of a core of the system of FIG. 1;

FIG. 3B is a bottom perspective view of the core of FIG. 3A;

FIG. 4A is an anterior view of the core of FIG. 3A;

FIG. 4B is a cross sectional lateral view of the core of FIG. 3A;

FIG. 4C is a lateral side view of the core of FIG. 3A;

FIG. 4D is a cross sectional anterior view of the core of FIG. 3A;

FIG. 5A is a top perspective view of a talar endplate of the system of FIG. 1;

FIG. 5B is a bottom perspective view of the talar endplate of FIG. 5A;

FIG. 6A is an anterior view of the talar endplate of FIG. 5A;

FIG. 6B is a cross sectional lateral view of the talar endplate of FIG. 5A;

FIG. 6C is a lateral side view of the talar endplate of FIG. 5A;

FIG. 6D is a cross sectional anterior view of the talar endplate;

FIG. 7 is an exploded view of the total ankle replacement system of FIG. 1;

FIG. 8A is an anterior view of the total ankle replacement system of FIG. 1 operatively assembled;

FIG. 8B is a cross sectional lateral view of the ankle replacement system of FIG. 1 operatively assembled;

FIG. 8C is a lateral view of the ankle replacement system of FIG. 1 operatively assembled;

FIG. 8D is a cross sectional anterior view of the ankle replacement system of FIG. 1 operatively assembled;

FIG. 9 is a front view of a set of cores;

FIG. 10 is a perspective view of the total ankle assembly of FIG. 1 with a neutral core implanted between a tibia and a talar bone;

FIG. 11A is a front view of the total ankle assembly of FIG. 1 with a 10 degree varus core implanted between a tibia and a talar bone;

FIG. 11B is a front view of the total ankle assembly of FIG. 10 with a neutral or 0 degree core implanted between a tibia and a talar bone; and

FIG. 11C is a front view of the total ankle assembly of FIG. 1 with a 10 degree valgus core implanted between a tibia and a talar bone.

### DETAILED DESCRIPTION

In this specification, standard medical directional terms are employed with their ordinary and customary meanings. Superior means toward the head. Inferior means away from the head. Anterior means toward the front. Posterior means toward the back. Medial means toward the midline, or plane of bilateral symmetry, of the body. Lateral means away from the midline of the body. Proximal means toward the trunk of the body. Distal means away from the trunk.

The present disclosure relates to systems, methods and kits for ankle anthroplasty, or in other words for replacing damaged and injured ankle joints with an artificial joint prosthesis. Those of skill in the art will recognize that the following description is merely illustrative of the principles of the technology, which may be applied in various ways to provide many different alternative embodiments. This description is made for the purpose of illustrating the general principles of this invention and is not meant to limit the inventive concepts in the appended claims.

In order to accommodate various patient deformities, it may be advantageous to have variation in the angle of articulation between the proximal end of the talus and the distal end of the tibia.

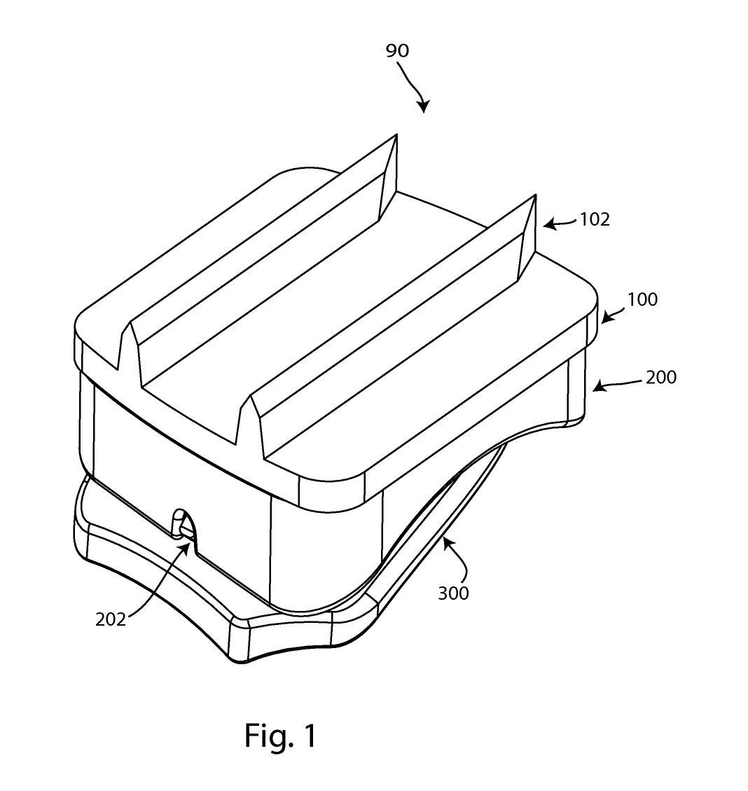

In one embodiment, an artificial ankle joint comprises a core, which may also be referred to as an articular insert or nucleus, beset on either side by endplates that may interact with the bones. Referring to FIGS. 1-3, an ankle replacement system is illustrated. System 90 may include a proximal bone-interfacing endplate 100, which may also be referred to as a tibial endplate, a core 200, or core component, and a distal bone-interfacing endplate 300, or talar plate, which may also be referred to as a talar endplate.

Referring to FIG. 1, a perspective view of an operatively assembled ankle replacement system is shown. Fins 102 are shown to protrude proximally from the tibial endplate 100, or tibial plate, to facilitate engagement with the bone, and may be coated in a bone growth enhancing material. It can also be seen in FIG. 1 that the core portion contains a slot 202, which will be shown to be congruent with a rib structure on the talar endplate.

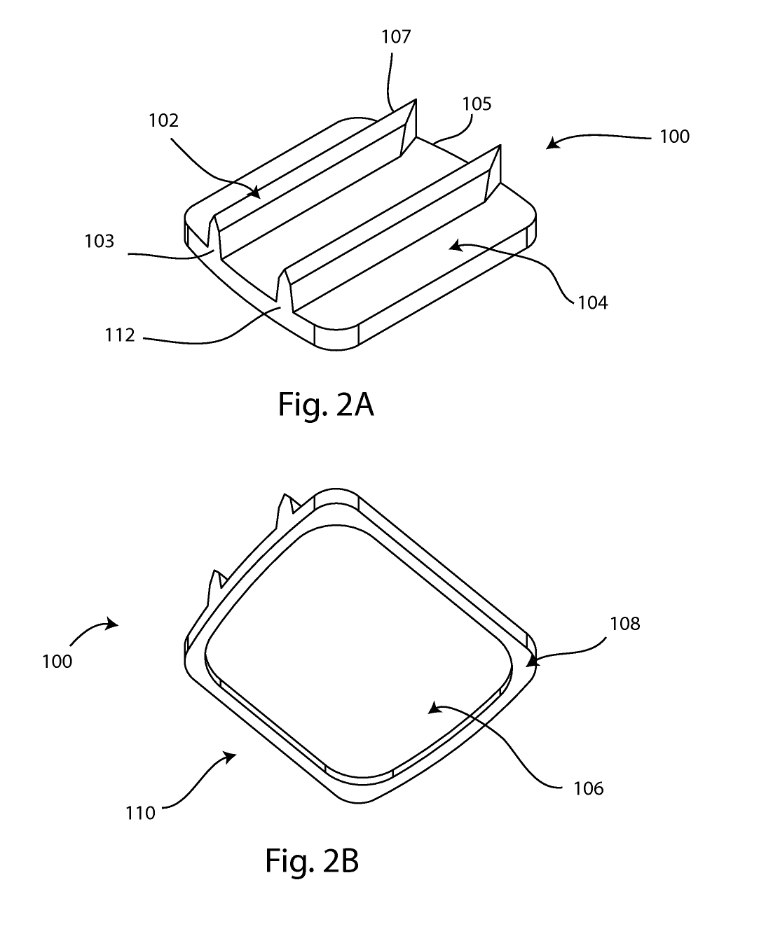

Referring to FIGS. 2A and 2B, a top perspective view and a bottom perspective view of the tibial endplate 100 is illustrated. The tibial endplate 100 may include a first bone-facing side 104, a second core-facing side 110 and an edge surface 112 extending between the two sides. The proximal, bone-facing side 104 of the tibial endplate may have a smooth surface, or may otherwise include surface roughening features, and may be provided with a bone growth enhancing media.

In FIG. 2A, at least one fin 102 is illustrated protruding from the proximal side 104 of the endplate 100, which fin may serve to facilitate interaction with the bone. The fin 102 may also be referred to as a keel, tooth, ridge or blade. In the example shown in FIG. 2A, two fins 102 are illustrated extending from a first end portion 103 to a second end portion 105 of the tibial endplate 100. In other examples, the fin or fins 102 may extend only partially between the first end 103 and the second end 105 of the endplate 100. The fins 102 are shown to be parallel to one another across the length of the tibial endplate 100, however, the fins 102 may have alternative orientations with respect to one another.

The fins 102 may include a sharpened edge 107 that is shaped to engage with a bone surface. The fins 102 may also have alternative surface geometries, such as rounded or otherwise contoured surfaces.

In FIG. 2A, the fins 102 may extend proximally perpendicularly to the first bone-facing surface 104. Additionally, in this example the fins 102 are shown to be integral with the bone-facing surface 104, however, the fins 102 may also be detachable from the tibial endplate 100.

FIG. 2B depicts the distal, core-facing side 110 of the endplate 100, which may be opposite to the first bone-facing surface 104 and may include a recessed surface 106 that is shaped to engage with a complementary feature on the core 200. The recessed surface may be at least partially encircled by a perimeter wall 108, or perimeter rim. The perimeter 108 of the recessed surface 106 may be of various sizes and shapes. The perimeter 108 may intersect the edge surface 112 of the tibial endplate 100. The endplate 100 may also contain a locking mechanism to secure the tibial endplate to the core.

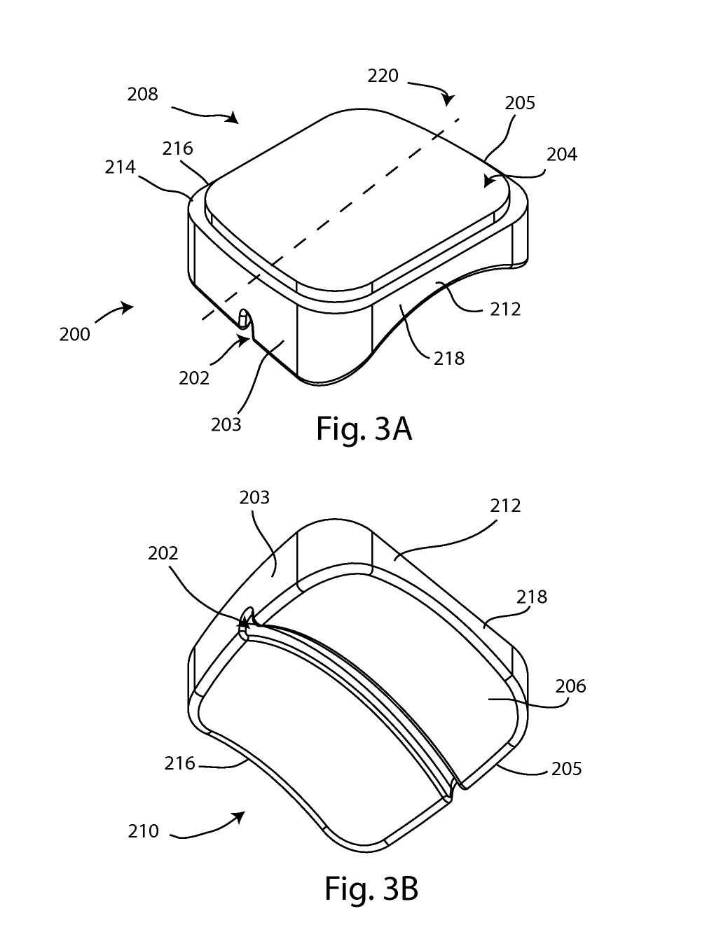

Referring to FIGS. 3A and 3B, different perspective views of the core 200 are illustrated. The core 200 may also be referred to as the articular insert or nucleus. The core may include a first endplate-facing surface 208, or tibial plate facing surface, a second endplate-facing surface 210, or talar plate facing surface, and an edge surface 212 that extends between the two endplate-facing surfaces. The edge surface 212 may be perpendicular to the first end plate-facing surface 208.

FIG. 3A shows that the first, or proximall or superior side of the core 200? contains a protruded surface 204, or protrusion, which is congruent with and complementary to the recessed area 106 of the tibial endplate 100. The protrusion may be at least partially encircled by a recessed perimeter surface 214. The core 200 may include a locking mechanism to secure the connection between the endplate and core section. For example, the core 200 may rigidly lock to the endplate 100 by an interference lock, Morse taper, or press fit.

FIG. 3B depicts the second, or distal, endplate-facing surface 210 of the core 200. The distal endplate-facing surface 210 may include a curved articular surface 206 and a slot 202. The slot 202 may also be referred to as a groove, cleft or a channel. The curved articular surface 206 may be smooth, and may be contoured to match a complementary contoured surface of the talar endplate 300. The edge surface 212 may include a first, or medial, wall 216 and a second, or lateral, wall 218 opposite the first wall. Wherein each wall 216, 218 may extend from the end plate-facing surface 208 to the curved articular surface 206.

The slot 202 may be rounded, as seen in FIG. 3B, or may have various other shapes and dimensions, such as chamfered or square edges. Here it is shown that the slot 202 extends entirely between a first end 203 and a second end 205 of the core 200. Alternatively, the slot may extend only a partial distance between the first end 203 and the second end 205.

It will be appreciated that the features of the recess 106 of the tibial plate 100 and the protrusion 204 of the core component 200 may be switched and have the same rigid locking. That is to say that a recess may be on the core component 200 and a protrusion on the tibial plate 100.

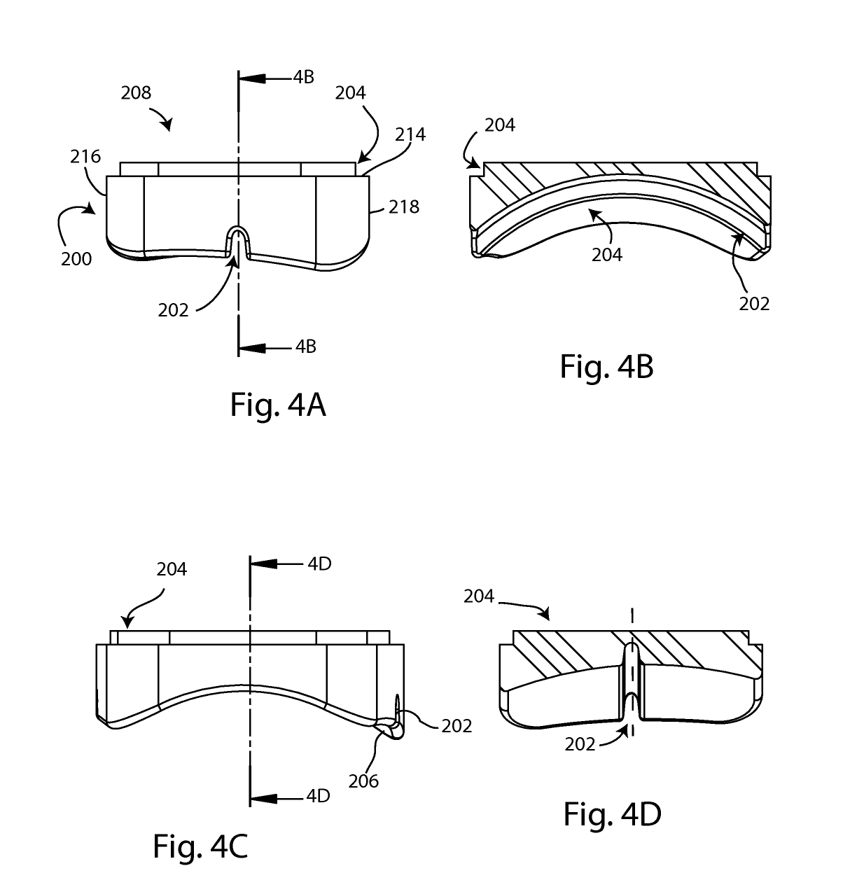

Referring to FIGS. 4A-4D, different views of the core 200 are illustrated. FIG. 4A is an anterior view of the core 200. The protruded surface 204 on the proximal endplate-facing side 208 can be seen extending from the recessed perimeter surface 214. On the distal endplate-facing side of the core 210, the slot 202 is illustrated as being substantially centrally located on the core 200, and having a symmetric cross section. The slot 202 may otherwise be located away from the center of the core 200, and the distal face 210 of the core may include more than one slot 202.

FIG. 4B provides a cross section of the core 200 of FIG. 4A from a lateral view through cross section line 4B-4B. The concave curvature of the distal side 210 of core 200 can be seen in FIG. 4B. FIG. 4C is a lateral view of the core 200, again showing the concave curvature of the distal endplate-facing side 210 of the core 200. FIG. 4D provides a cross section of the core 200 of FIG. 4C from an anterior view through cross section line 4D-4D. The slot 202 can be clearly seen in FIG. 4D.

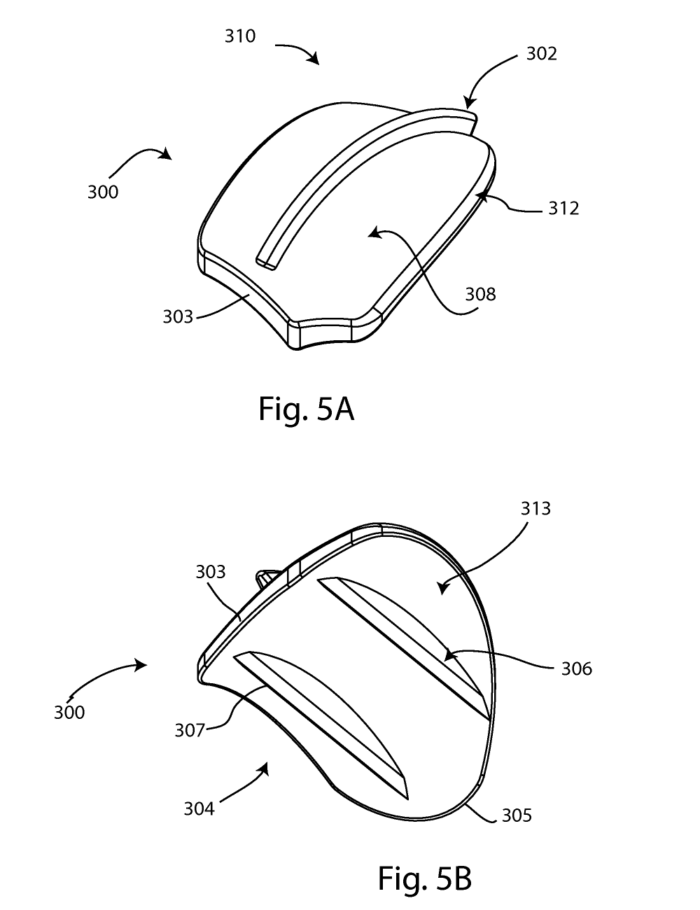

FIGS. 5A and 5B provide different perspective views of the talar endplate 300, which is located distal to the core 200 when the total ankle assembly is operatively arranged as illustrated in FIG. 1. The talar endplate comprises a proximal core-facing surface 310, a distal bone-facing side 304 and an edge surface 312 connecting the proximal and distal surfaces. The bone-facing surface 304 may also be provided with a biocompatible bone growth enhancing media.

FIG. 5A provides a top perspective view of the talar endplate 300. The proximal surface 310 may be an articulated, contoured surface that is congruent with the curvature of the distal articulating surface 206 of the core 200. The proximal surface 310 may be smooth, or may contain a variety of surface-roughening features.

A rib 302 is depicted protruding proximally from the proximal surface 308, which may be congruent with the slot 202 on the distal endplate-facing side of the core 200. The rib 302, which may also be referred to as a rail, may extend at least partially between a first end portion 303 and a second end portion 305 of talar endplate 300. The talar endplate 300 may also include more than one rib 302 to engage the core 200. An equal number of ribs and slots may be provided on complementary talar endplates and cores.

The rib 302 may be shaped such that it can slide within the slot 202, providing for limited joint articulation and limited degrees of freedom when the ankle system 90 is assembled. In one example, the rib and slot may be closely fitted so that articulation is limited to a direction established by the rib and slot. In another example, the slot may be wider than the rib so that articulation may include rotation about the long axis of the tibia or in varus/valgus directions.

FIG. 5B provides a bottom perspective view of talar endplate 300. FIG. 5B illustrates the presence of endplate teeth, also referred to as keels, 306, 307, on the distal bone-facing side 304 of the talar plate 300. These teeth may have a thin, sharpened edge 307 to facilitate interaction of the ankle replacement system 90 with the proximal section of the talar bone. The teeth may also be of various other shapes and dimensions.

As shown in FIG. 5B, distal bone-facing surface 304 may include a resurfacing talar surface 313. FIG. 5B depicts the resurfacing talar surface 313 as a concave curved surface, however, it may also be flat or convexly shaped, depending on the nature of the surgical procedure and on the patient anatomy.

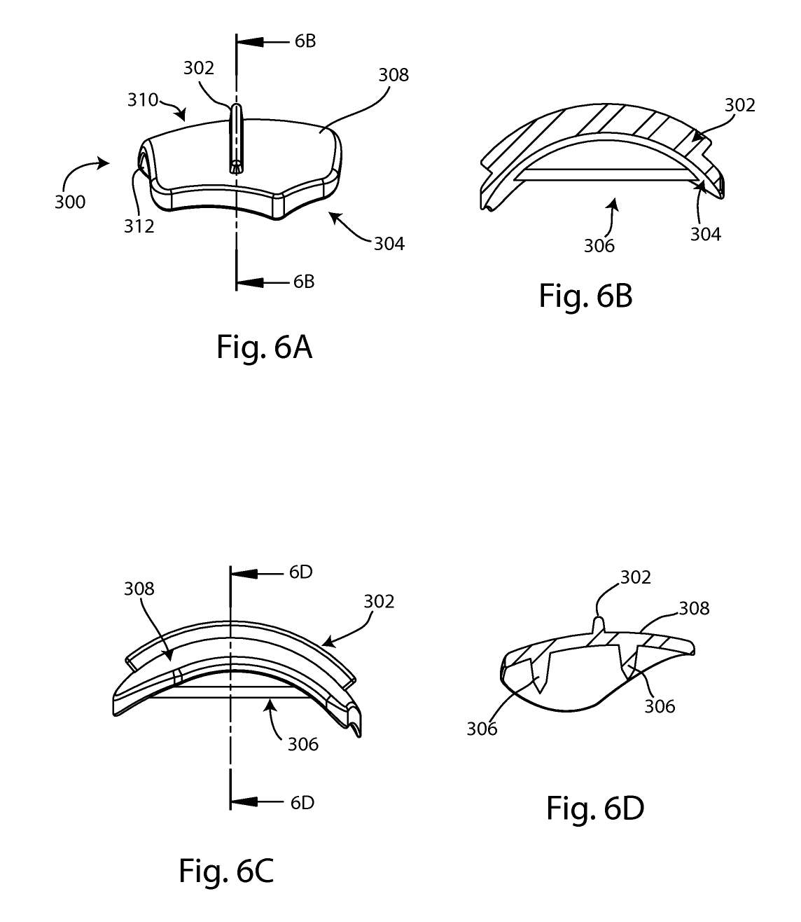

FIGS. 6A-6D provide additional views and cross-sections of the talar endplate 300. FIG. 6A provides a front view of the talar endplate 300 and depicts the convex curvature on the proximal core-facing side 310 of the talar endplate 300, which is congruent with the concave curvature of the distal endplate-facing side 210 of the core 200. Also shown in FIG. 6A is the rib 302 that protrudes from the articular surface 308 of the talar endplate. FIG. 6B provides a cross-section of the talar plate 300 of FIG. 6A through section line 6B-6B and shows one of the endplate teeth 306 that protrude from the resurfacing surface 313. FIG. 6C is a lateral view of the talar endplate 300. FIG. 6D provides a cross section of the talar endplate of 8C through section line 6D-6D and gives another view of the top surface 308 of the talar endplate and protruded rib 302 section, as well as the two teeth extensions 306 from the distal bone-facing side 304 of the talar endplate 300.

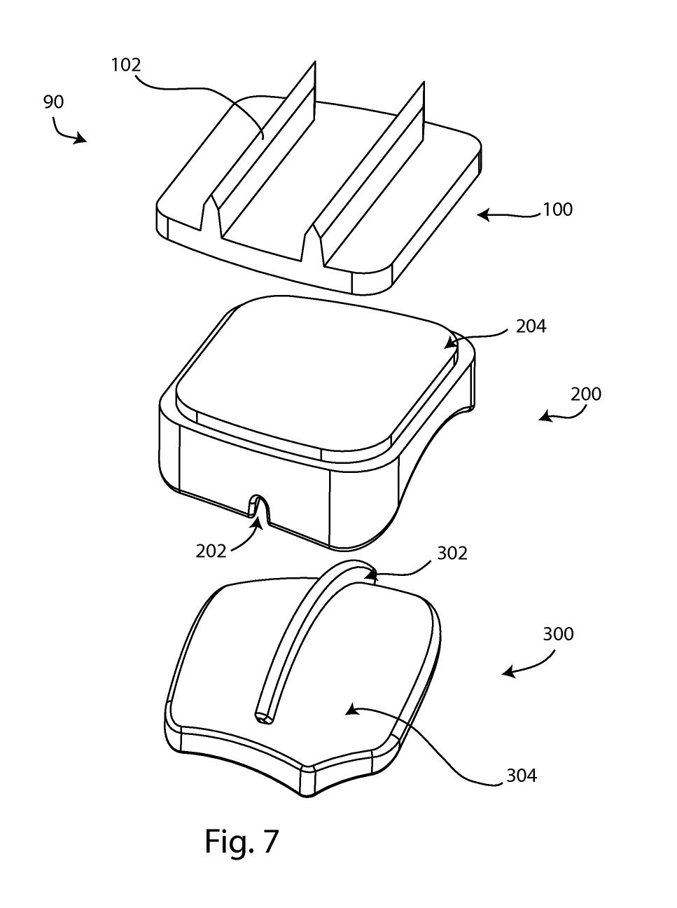

Referring to FIG. 7, an exploded perspective view of the three primary components of system 90 is illustrated. When system 90 is operatively assembled, the tibial endplate 100 is located proximal to the core 200. The recessed surface 106 on the distal surface 110 of the tibial endplate 100 receives the protruded surface 204 of the core 200 and rigidly locks the core 200 to the endplate 100. The talar endplate 300 sits distal to the core 200. The rib structure 302 is received by the slot feature 202 located on the distal side 210 of the core 200. The curvature of the proximal surface 308 is congruent with the distal side 210 of the core 200. The talar endplate 300 articulates congruently with the core 200, at least by sliding along a direction established by the rib 302 and the slot 202. The first end portions 103, 203, 303 of the tibial endplate 100, the core 200, and the talar endplate 300, respectively, all face the same way.

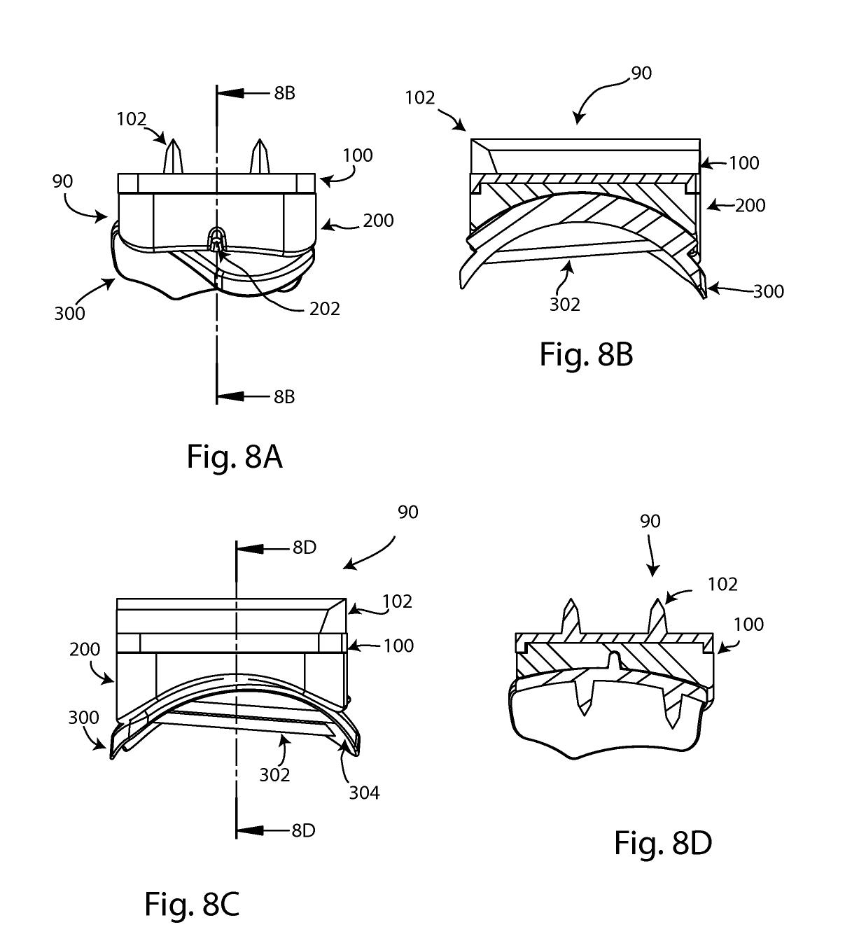

FIGS. 8A-8D provide several views and cross sections of the assembled ankle replacement. FIG. 8A provides a front view of the assembled ankle replacement. The core piece 200 is operatively assembled with a tibial endplate 100 and a talar endplate 300. FIG. 8B provides a cross section of the ankle assembly of FIG. 8A through section line 8B-8B. The orientation of the three components is shown as assembled. FIG. 8C provides a lateral view of the assembled ankle replacement system. FIG. 8D provides a cross section of the ankle replacement system through section line 8D-8D.

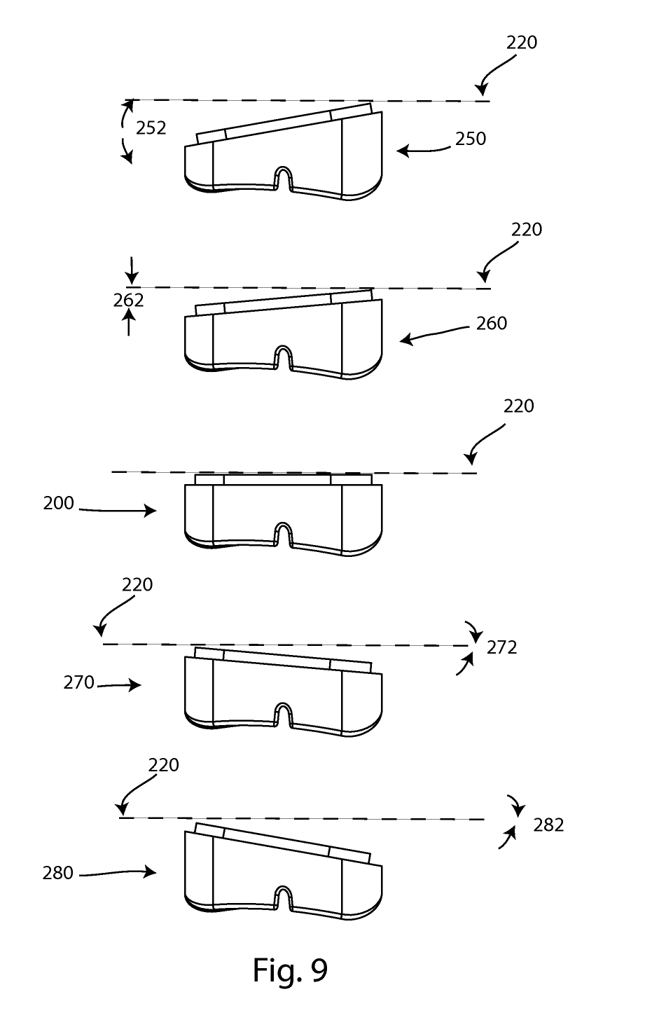

FIG. 9 provides a front view of a set of cores with different varus and valgus orientations. Shown at the top of FIG. 9, core 250 is angled in a valgus orientation at angle 252 relative to the horizontal line 220. The core 250 in a valgus orientation includes a first, or medial, 216 wall height that is shorter than a second, or lateral, wall 218 height. Core 260 is also angled in a valgus orientation at angle 262 relative to the horizontal line 220, wherein angle 262 is less than 252. Similar to core 250, the core 260 in a valgus orientation includes a first wall, or medial wall, 216 height that is shorter than a second wall, or lateral wall, 218 height; however the difference is less between the first wall 216 height to the second wall 218 height in core 260 than in core 250. In each of the cores 250, 260 a plane of the tibial plate facing surface forms an acute angle relative to a horizontal line. Core 200 is oriented as previously described in a neutral position, with the elevated top portion flush with the horizontal line 220. Core 270 is angled in a varus orientation, at angle 272 relative to the horizontal line 220. The core 270 in a varus orientation includes a first wall, or medial wall, 216 height that is longer than a second wall, or lateral wall, 218 height. Core 280 is angled in a varus orientation at angle 282, wherein angle 282 is greater than 272 relative to the horizontal line 220. Similar to core 270, the core 280 in a varus orientation includes a first wall 216 height that is longer than a second wall 218 height; however the difference is more between the second wall 218 height to the first wall 216 height in core 280 than in core 270. In each of the cores 270, 280 a plane of the tibial plate facing surface forms an acute angle relative to a horizontal line. These are some examples of a comprehensive set or kit of cores which may be interchangeably used in the disclosed total ankle replacement.

In the present system, a varus or valgus deformity of an ankle joint may be corrected by selecting and inserting a core which compensates for, or neutralizes, the deformity. The bone resections on the tibia may be made with reference to the tibia alone, and the cuts may be aligned so that a minimal amount of bone is resected. In one example, the tibial resections may be made with reference to the distal tibial articular surface, regardless of the orientation of the distal tibial articular surface. In a similar manner, the bone resections on the proximal talus may be made with reference to the talus alone. In one example, the talar resections may be made with reference to the proximal talar articular surface, regardless of its orientation. In this arrangement, a suitable core may be interposed between the endplates to compensate for deformity and restore a neutral orientation between the tibia and talus.

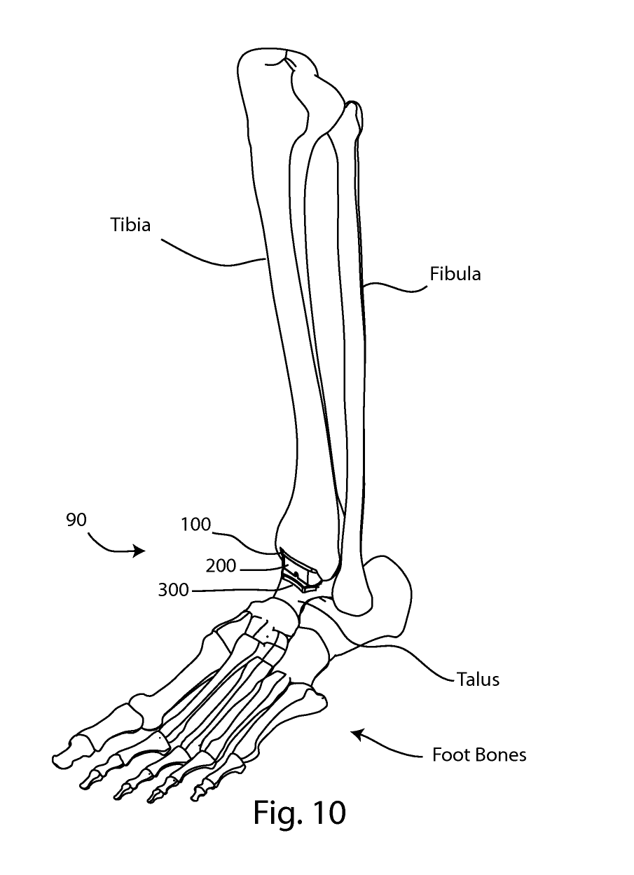

FIG. 10 provides a perspective view of the disclosed ankle replacement system 90 with a neutral core implanted between the tibia and talar bones.

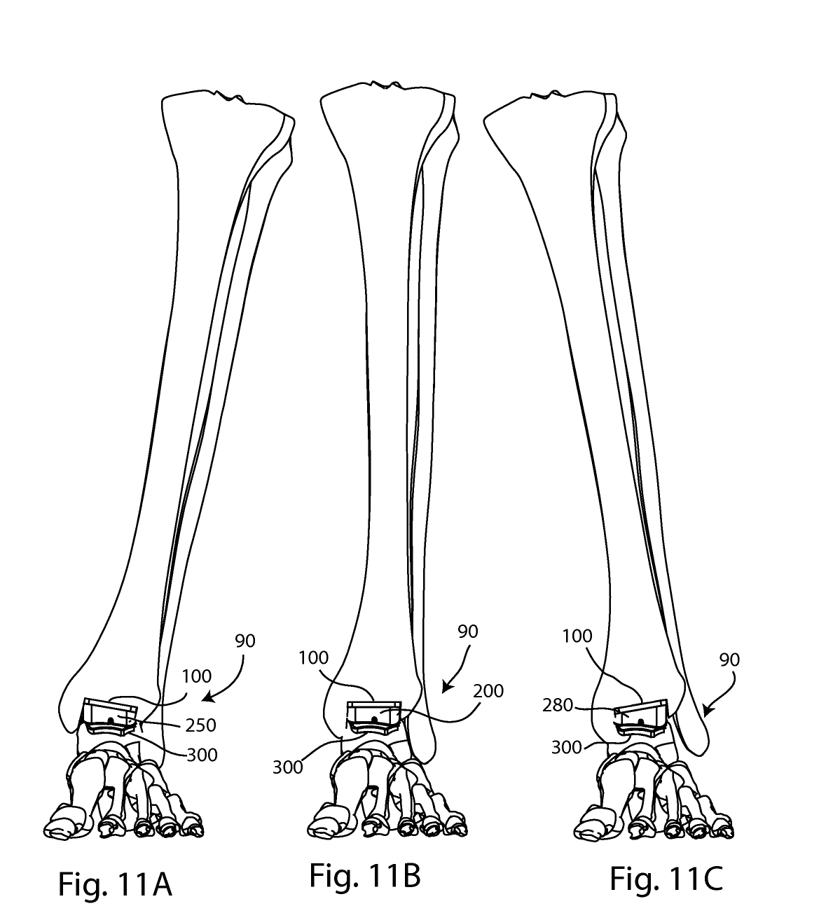

FIGS. 11A-11C shows the disclosed total ankle replacement as it would appear relative to the tibia and talar bones in different varus-valgus formations. FIG. 11A shows the disclosed ankle replacement in a 10 degree varus formation, FIG. 11B shows the disclosed ankle replacement in a neutral formation and FIG. 11C shows the disclosed ankle replacement in a 10 degree valgus formation.

1. A system comprising:
a tibial plate comprising a tibial facing surface and an opposite core facing surface, wherein the tibial facing surface comprises at least one fin outwardly projecting and insertable into a bone;
a core component comprising a tibial plate facing surface, an opposite talar plate facing surface, a first wall and a second wall opposite the first wall, wherein the first and second walls extend from the tibial plate facing surface to the opposite talar plate facing surface, wherein one of the first or second walls is shorter than one of the other first or second walls; and
a talar plate comprising a core facing surface and an opposite talar facing surface, wherein the opposite core facing surface comprises a rail outwardly projecting, and wherein the talar facing surface comprises at least one keel outwardly projecting.

2. The system of claim 1, wherein one of the first or second wall is shorter than the other first or second wall in a varus orientation.

3. The system of claim 2, wherein the core facing surface comprises a recess defined by a perimeter rim on the tibial plate.

4. The system of claim 3, wherein the tibial plate facing surface comprises a protrusion, wherein the recess and the protruding surface form a complementary fit locking the core component to the tibial plate.

5. The system of claim 1, wherein one of the first or second wall is shorter than the other first or second wall in a valgus orientation.

6. The system of claim 5, wherein the core facing surface comprises a recess defined by a perimeter rim on the tibial plate.

7. The system of claim 6, wherein the tibial plate facing surface comprises a protrusion, wherein the recess and the protruding surface form a complementary fit locking the core component to the tibial plate.

8. A system comprising:
a tibial plate comprising a bone facing surface and a core facing surface, wherein the bone facing surface comprises at least one fin;
a core component comprising a tibial plate facing surface and a talar plate facing surface, wherein the talar plate facing surface is a curved articular surface, and wherein the tibial plate facing surface comprises a protrusion configured to engage with the tibial plate core facing surface in a complementary fit; and
a talar plate substantially curved and congruent with the curvature of the talar plate facing surface of the core component.

9. The system of claim 8, wherein the talar plate comprises substantially uniform thickness.

10. The system of claim 9, wherein the curvature of the talar plate is concave.

11. The system of claim 10, wherein the talar plate comprises a superior core facing surface and a talar facing surface, wherein the superior core facing surface comprises a rail extending superiorly, and wherein the talar facing surface comprises at least one keel extending inferiorly.

12. The system of claim 11, wherein the core component further comprises first wall and a second wall opposite the first wall, wherein the first and second walls extend from the tibial plate facing surface to the opposite talar plate facing surface, wherein one of the first or second walls is shorter than one of the other first or second walls.

13. The system of claim 12, wherein the core facing surface comprises a recess defined by a perimeter rim on the tibial plate, wherein the recess and the protruding surface form a complementary fit locking the core component to the tibial plate.

14. The system of claim 8, wherein the tibial plate facing surface of the core component is substantially horizontal.

15. The system of claim 14, wherein the core facing surface comprises a recess defined by a perimeter rim on the tibial plate, wherein the recess and the protrusion form a complementary fit.

16. A system comprising:
a tibial plate comprising a tibial facing surface and an inferior core facing surface, wherein the bone facing comprises at least one fin;
a core component comprising a tibial plate facing surface and a talar plate facing surface, wherein the talar plate facing surface comprises a groove configured to engage the talar plate, and wherein the tibial plate facing surface comprises a protrusion configured to engage with the core facing surface of the tibial plate in a complementary fit; and
a talar plate comprising a superior core facing surface and a talar facing surface, wherein the superior core facing surface comprises a rail extending superiorly, and wherein the talar facing surface comprises at least one keel extending inferiorly, the keel insertable into a bone.

17. The system of claim 16, wherein the core facing surface comprises a recess defined by a perimeter rim on the tibial plate, wherein the recess and the protrusion form a complementary fit.

18. The system of claim 17, wherein the groove extends in an anterior-posterior direction from an anterior end of the core to a posterior end of the core.

19. The system of claim 18, wherein the rail extends in an anterior-posterior direction and is configured to engage the groove to limit articulation in a direction established by the rail and the groove.

20. The system of claim 19, wherein the core component further comprises first wall and a second wall opposite the first wall, wherein the first and second walls extend from the tibial plate facing surface to the opposite talar plate facing surface, wherein one of the first or second walls is shorter than one of the other first or second walls.
